SEQUENCE LISTING (4 sequences)

SEQ ID NO: 1 - DNA, 15418 bp, Homo sapiens
gcggccgcgagctctaatacgactcactatagggcgtcgactcgatcaatggaagatgaggcattgccgaagaaaagattaatggatttgaacacacagcaacagaaactacatgaagtgaaacacaggaaaaaaaagataaagaaacgaaaagaaaagggcatcagtgagcttcagcagaagttccatcggccttacatatgtgtaagcagaggccctgtaggagcagaggcagggggaaaatactttaagaaataatgtctaaaagtttttcaaatatgaggaaaaacataaaaccacagatccaagaagctcaacaaaacaaagcacaagaaacaggaagaaattaaaagttatatcacagtcaaattgctgaaaaccagcaacaaagagaatatcttaagagtatcagaggaaaagagattaatgacaggccaagaaacaatgaaaacaatacagatttcttgtaggaaacacaagacaaaagacattttttaaaaccaaaaggaaaaaaaatgctacattaaaatgttttttacccactgaaagtatatttcaaaacatattttaggccaggcttggtggctcacacctgtaatcccagcactttgggaggccaaggtgggtggatcgcttaaggtcaggagttcgagaccagcctggccaatatagcgaaaccccatctgtactaaaaacacaaaaattagctgggtgtggtgacacatgcctgtaatcccaggtactcaggaggctaaggcaggagaattgcttgaactgggaggcagaggtggtgagccaagattgcaccagtgcactccagccttggtgacagagtgaaactccatctcaaaaacaaacaaacaaaatacatatacataaatatatatgcacatatatatacatatataaatatatatacacatatataaatctatatacatatatacatatatacacatatataaatctatatacatatatatacatatataatatatttacatatataaatatatacatatataaatatacatatataaatacatatataaatatacatatataaatatacatatataaatatacatatataaatatatacatatataaatatacatatataaatatatatacatatataaatatataaatatacaagtatatacaaatatatacatatataaatgtatatacgtatatacatatatatataaatatataaaaaaacttttggctgggcacctttccaaatctcatggcacatataagtctcatggtaacctcaaataaaaaaacatataacagatacaccaaaaataaaaaccaataaattaaatcatgccaccagaagaaattaccttcactaaaaggaacacaggaaggaaagaaagaaggaagagaagaccatgaaacaaccagaaaacaaacaacaaaacagcaggagtaattcctgacttatcaataataatgctgggtgtaaatggactaaactctccaatcaaaagacatagagtggctgaatggacgaaaaaaacaagactcaataatctgttgcctacaagaatatacttcacctataaagggacacatagactgaaaataaaaggaaggaaaaatattctatgcaaatggaaaccaaaaaaagaacagaactagctacacttatatcagacaaaatagatttcaagacaaaaagtacaaaaagagacaaagtaattatataataataaagcaaaaagatataacaattgtgaatttatatgcgcccaacactgggacacccagatatatacagcaaatattattagaactaaggagagagagagatccccatacaataatagctggagacttcaccccgcttttagcattggacagatcatccagacagaaaatcaaccaaaaaattggacttaatctataatatagaacaaatgtacctaattgatgtttacaagacatttcatccagtagttgcagaatatgcattttttcctcagcatatggatcattctcaaggatagaccatatattaggccacagaacaagccattaaaaattcaaaaaaattgagccaggcatgatggcttatgcttgtaattacagcactttggggagggtgaggtgggaggatgtcttgagtacaggagtttgagaccagcctgggcaaaatagtgagaccctgtctctacaaacttttttttttaattagccaggcatagtggtgtgtgcctgtagtcccagctacttaggaggctgaagtgggaggatcacttgagcccaagagttcaaggctacggtgagccatgattgcaacaccacacaccagccttggtgacagaatgagaccctgtctcaaaaaaaaaaaaaaaaattgaaataatataaagcatcttctctggccacagtggaacaaaaccagaaatcaacaacaagaggaattttgaaaactatacaaacacatgaaaattaaacaatatacttctgaataaccagtgagtcaatgaagaaattaaaaaggaaattgaaaaatttatttaagcaaatgataacggaaacataacctctcaaaacccacggtatacagcaaaagcagtgctaagaaggaagtttatagctataagcagctacatcaaaaaagtagaaaagccaggcgcagtggctcatgcctgtaatcccagcactttgggaggccaaggcgggcagatcgcctgaggtcaggagttcgagaccagcctgaccaacacagagaaaccttgtcgctactaaaaatacaaaattagctgggcatggtggcacatgcctgtaatcccagctactcgggaggctgaggcaggataaccgcttgaacccaggaggtggaggttgcggtgagccgggattgcgccattggactccagcctgggtaacaagagtgaaaccctgtctcaagaaaaaaaaaaaagtagaaaaacttaaaaatacaacctaatgatgcaccttaaagaactagaaaagcaagagcaaactaaacctaaaattggtaaaagaaaagaaataataaagatcagagcagaaataaatgaaactgaaagataacaatacaaaagatcaacaaaattaaaagttggttttttgaaaagataaacaaaattgacaaacctttgcccagactaagaaaaaaggaaagaagacctaaataaataaagtcagagatgaaaaaagagacattacaactgataccacagaaattcaaaggatcactagaggctactatgagcaactgtacactaataaattgaaaaacctagaaaaaatagataaattcctagatgcatacaacctaccaagattgaaccatgaagaaatccaaagcccaaacagaccaataacaataatgggattaaagccataataaaaagtctcctagcaaagagaagcccaggacccaatggcttccctgctggattttaccaatcatttaaagaagaatgaattccaatcctactcaaactattctgaaaaatagaggaaagaatacttccaaactcattctacatggccagtattaccctgattccaaaaccagacaaaaacacatcaaaaacaaacaaacaaaaaaacagaaagaaagaaaactacaggccaatatccctgatgaatactgatacaaaaatcctcaacaaaacactagcaaaccaaattaaacaacaccttcgaaagatcattcattgtgatcaagtgggatttattccagggatggaaggatggttcaacatatgcaaatcaatcaatgtgatacatcatcccaacaaaatgaagtacaaaaactatatgattatttcactttatgcagaaaaagcatttgataaaattctgcacccttcatgataaaaaccctcaaaaaaccaggtatacaagaaacatacaggccaggcacagtggctcacacctgcgatcccagcactctgggaggccaaggtgggatgattgcttgggcccaggagtttgagactagcctgggcaacaaaatgagacctggtctacaaaaaacttttttaaaaaattagccaggcatgatggcatatgcctgtagtcccagctagtctggaggctgaggtgggagaatcacttaagcctaggaggtcgaggctgcagtgagccatgaacatgtcactgtactccagcctagacaacagaacaagaccccactgaataagaagaaggagaaggagaagggagaaaggagggagaagggaggaggaggagaaggaggaggtggaggagaagtggaaggggaaggggaagggaaagaggaagaagaagaaacatatttcaacataataaaagccctatatgacagaccgaggtagtattatgaggaaaaactgaaagcctttcctctaagatctggaaaatgacaagggcccactttcaccactgtgattcaacatagtactagaagtcctagctagagcaatcagataagagaaagaaataaaaggcatccaaactggaaaggaagaagtcaaattatcctgtttgcagatgatatgatcttatatctggaaaagacttaagacaccactaaaaaactattagagctgaaatttggtacagcaggatacaaaatcaatgtacaaaaatcagtagtatttctatattccaacagcaaacaatctgaaaaagaaaccaaaaaagcagctacaaataaaattaaacagctaggaattaaccaaagaagtgaaagatctctacaatgaaaactataaaatattgataaaagaaattgaagagggcacaaaaaaagaaaagatattccatgttcatagattggaagaataaatactgttaaaatgtccatactacccaaagcaatttacaaattcaatgcaatccctattaaaatactaatgacgttcttcacagaaatagaagaaacaattctaagatttgtacagaaccacaaaagacccagaatagccaaagctatcctgaccaaaaagaacaaaactggaagcatcacattacctgacttcaaattatactacaaagctatagtaacccaaactacatggtactggcataaaaacagatgagacatggaccagaggaacagaatagagaatccagaaacaaatccatgcatctacagtgaactcatttttgacaaaggtgccaagaacatactttggggaaaagataatctcttcaataaatggtgctggaggaactggatatccatatgcaaaataacaatactagaactctgtctctcaccatatacaaaagcaaatcaaaatggatgaaaggcttaaatctaaaacctcaaactttgcaactactaaaagaaaacaccggagaaactctccaggacattggagtgggcaaagacttcttgagtaattccctgcaggcacaggcaaccaaagcaaaaacagacaaatgggatcatatcaagttaaaaagcttctgcccagcaaaggaaacaatcaacaaagagaagagacaacccacagaatgggagaatatatttgcaaactattcatctaacaaggaattaataaccagtatatataaggagctcaaactactctataagaaaaacacctaataagctgattttcaaaaataagcaaaagatctgggtagacatttctcaaaataagtcatacaaatggcaaacaggcatctgaaaatgtgctcaacaccactgatcatcagagaaatgcaaatcaaaactactatgagagatcatctcaccccagttaaaatggcttttattcaaaagacaggcaataacaaatgccagtgaggatgtggataaaaggaaacccttggacactgttggtgggaatggaaattgctaccactatggagaacagtttgaaagttcctcaaaaaactaaaaataaagctaccatacagcaatcccattgctaggtatatactccaaaaaagggaatcagtgtatcaacaagctatctccactcccacatttactgcagcactgttcatagcagccaaggtttggaagcaacctcagtgtccatcaacagacgaatggaaaaagaaaatgtggtgcacatacacaatggagtactacgcagccataaaaaagaatgagatcctgtcagttgcaacagcatggggggcactggtcagtatgttaagtgaaataagccaggcacagaaagacaaacttttcatgttctcccttacttgtgggagcaaaaattaaaacaattgacatagaaatagaggagaatggtggttctagaggggtgggggacagggtgactagagtcaacaataatttattgtatgttttaaaataactaaaagagtataattgggttgtttgtaacacaaagaaaggataaatgcttgaaggtgacagataccccatttaccctgatgtgattattacacattgtatgcctgtatcaaaatatctcatgtatgctatagatataaaccctactatattaaaaattaaaattttaatggccaggcacggtggctcatgtccataatcccagcactttgggaggccgaggcggtggatcacctgaggtcaggagtttgaaaccagtctggccaccatgatgaaaccctgtctctactaaagatacaaaaattagccaggcgtggtggcacatacctgtagtcccaactactcaggaggctgagacaggagaattgcttgaacctgggaggcggaggttgcagtgagccgagatcatgccactgcactgcagcctgggtgacagagcaagactccatctcaaaacaaaaacaaaaaaaagaagattaaaattgtaatttttatgtaccgtataaatatatactctactatattagaagttaaaaattaaaacaattataaaaggtaattaaccacttaatctaaaataagaacaatgtatgtggggtttctagcttctgaagaagtaaaagttatggccacgatggcagaaatgtgaggagggaacagtggaagttactgttgttagacgctcatactctctgtaagtgacttaattttaaccaaagacaggctgggagaagttaaagaggcattctataagccctaaaacaactgctaataatggtgaaaggtaatctctattaattaccaataattacagatatctctaaaatcgagctgcagaattggcacgtctgatcacaccgtcctctcattcacggtgctttttttcttgtgtgcttggagattttcgattgtgtgttcgtgtttggttaaacttaatctgtatgaatcctgaaacgaaaaatggtggtgatttcctccagaagaattagagtacctggcaggaagcaggtggctctgtggacctgagccacttcaatcttcaagggtctctggccaagacccaggtgcaaggcagaggcctgatgacccgaggacaggaaagctcggatgggaaggggcgatgagaagcctgcctcgttggtgagcagcgcatgaagtgcccttatttacgctttgcaaagattgctctggataccatctggaaaaggcggccagcgggaatgcaaggagtcagaagcctcctgctcaaacccaggccagcagctatggcgcccacccgggcgtgtgccagagggagaggagtcaaggcacctcgaagtatggcttaaatctttttttcacctgaagcagtgaccaaggtgtattctgagggaagcttgagttaggtgccttctttaaaacagaaagtcatggaagcacccttctcaagggaaaaccagacgcccgctctgcggtcatttacctctttcctctctccctctcttgccctcgcggtttctgatcgggacagagtgacccccgtggagcttctccgagcccgtgctgaggaccctcttgcaaagggctccacagacccccgccctggagagaggagtctgagcctggcttaataacaaactgggatgtggctgggggcggacagcgacggcgggattcaaagacttaattccatgagtaaattcaacctttccacatccgaatggatttggattttatcttaatattttcttaaatttcatcaaataacattcaggagtgcagaaatccaaaggcgtaaaacaggaactgagctatgtttgccaaggtccaaggacttaataaccatgttcagagggatttttcgccctaagtactttttattggttttcataaggtggcttagggtgcaagggaaagtacacgaggagaggactgggcggcagggctatgagcacggcaaggccaccggggagagagtccccggcctgggaggctgacagcaggaccactgaccgtcctccctgggagctgccacattgggcaacgcgaaggcggccacgctgcgtgtgactcaggaccccataccggcttcctgggcccacccacactaacccaggaagtcacggagctctgaacccgtggaaacgaacatgacccttgcctgcctgcttccctgggtgggtcaagggtaatgaagtggtgtgcaggaaatggccatgtaaattacacgactctgctgatggggaccgttccttccatcattattcatcttcacccccaaggactgaatgattccagcaacttcttcgggtgtgacaagccatgacaacactcagtacaaacaccactcttttactaggcccacagagcacggcccacacccctgatatattaagagtccaggagagatgaggctgctttcagccaccaggctggggtgacaacagcggctgaacagtctgttcctctagactagtagaccctggcaggcactcccccagattctagggcctggttgctgcttcccgagggcgccatctgccctggagactcagcctggggtgccacactgaggccagccctgtctccacaccctccgcctccaggcctcagcttctccagcagcttcctaaaccctgggtgggccgtgttccagcgctactgtctcacctgtcccactgtgtcttgtctcagcgacgtagctcgcacggttcctcctcacatggggtgtctgtctccttccccaacactcacatgcgttgaagggaggagattctgcgcctcccagactggctcctctgagcctgaacctggctcgtggcccccgatgcaggttcctggcgtccggctgcacgctgacctccatttccaggcgctccccgtctcctgtcatctgccggggcctgccggtgtgttcttctgtttctgtgctcctttccacgtccagctgcgtgtgtctctgtccgctagggtctcggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcacttttctgcccccctccctctggaacacagagtggcagtttccacaagcactaagcatcctcttcccaaaagacccagcattggcacccctggacatttgccccacagccctgggaattcacgtgactacgcacatcatgtacacactcccgtccacgaccgacccccgctgttttattttaatagctacaaagcagggaaatccctgctaaaatgtcctttaacaaactggttaaacaaacgggtccatccgcacggtggacagttcctcacagtgaagaggaacatgccgtttataaagcctgcaggcatctcaagggaattacgctgagtcaaaactgccacctccatgggatacgtacgcaacatgctcaaaaagaaagaatttcaccccatggcaggggagtggttggggggttaaggacggtgggggcagcagctgggggctactgcacgcaccttttactaaagccagtttcctggttctgatggtattggctcagttatgggagactaaccataggggagtggggatgggggaacccggaggctgtgccatctttgccatgcccgagtgtcctgggcaggataatgctctagagatgcccacgtcctgattcccccaaacctgtggacagaacccgcccggccccagggcctttgcaggtgtgatctccgtgaggaccctgaggtctgggatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaggctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcgagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggggtggggttcaccatgttggccaggctggtctcgaacttctgacctcagatgatccacctgcctctgcctcctaaagtgctgggattacaggtgtgagccaccatgcccagctcagaatttactctgtttagaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcgatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacccgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggtgaggaggtggtggccgtcgagggcccaggccccagagctgaatgcagtaggggctcagaaaagggggcaggcagagccctggtcctcctgtctccatcgtcacgtgggcacacgtggcttttcgctcaggacgtcgagtggacacggtgatcgagtcgactccctttagtgagggttaattgagctcgcggccgc

SEQ ID NO: 2 - DNA, 1481 bp, Homo sapiens
  CDS (127)..(1080)
ccgggagcggagagcggaccccagagagccctgagcagccccaccgccgccgccggcctagttaccatcacaccccgggaggagccgcagctgccgcagccggccccagtcaccatcaccgcaaccatgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgaggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcccgctcccgaggctgagcagggcggggctgagtaaatgccggcttaccatctctaccatcatccggtttagtcatccaacaagaagaaatatgaaattccagcaataagaaatgaacaaaagattggagctgaagacctaaagtacttgctttttgccgtttgcaaccagataaatagaactatctgcattatctatgcagcatggggtttatattttactaagacgctctttggtatacaacggttttaaaagcctggttttctcaatacgccttaaaggttttaaattgtttcatatctggtcaagttgagatttttaagaacttcatttttaatttgtaataaaagtttacaacttgattttttcaaaaaagtcaacaaactgcaagcacctgttaataaaggtcttaaataat

SEQ ID NO: 3 - protein, 317 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGEEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSRSRG

SEQ ID NO: 4 - DNA, 3360 bp, Human cytomegalovirus
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgatggagtcctctgccaagagaaagatggaccctgataatcctgacgagggcccttcctccaaggtgccacggtacgtgtcggggtttgtgccccccctttttttttaataaaattgtattaatgttatatacatatctcctgtatgtgacccatgtgcttatgactctatttctcatgtgtttaggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggatacttatatgtgttgttatcctcctctacagtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaaactattgtatatatatatcagttactgttatggatcccacgtcactattgtatactctatattatactctatgttatactctgtaatcctactcaataaacgtgtcacgcctgtgaaaccgt

(19) United States
(12) Patent Application Publication
(10) Pub. No.: US 2003/0095989 A1
(43) Pub. Date: May 22, 2003

(54) Chimeric cytolytic viruses for cancer treatment

(76) Inventors: John M. Irving, San Mateo, CA (US); Jane S. Lebkowski, Portola Valley, CA (US)

Correspondence Address: GERON CORPORATION, 230 CONSTITUTION DRIVE, MENLO PARK, CA, 94025

(21) Appl. No.: 10/023,969

(22) Filed: Dec. 17, 2001

Related U.S. Application Data
(60) Provisional application No. 60/256,418, filed on Dec. 18, 2000.

(51) Int. Cl. A61K 48/00; C12Q 1/70; C12N 7/01; C12N 5/08; A01N 63/00; A61K 39/12; A61K 39/245; A61K 39/25; A61K 39/23; A61K 39/235; C12N 7/00; C12N 15/00; C12N 15/09; C12N 15/63; C12N 15/70; C12N 15/74; C12N 5/00; C12N 5/02

(52) U.S. Cl. 424/233.1; 424/93.6; 435/235.1; 435/5; 435/366; 424/230.1; 424/199.1; 435/320.1; 435/325

(57) ABSTRACT

Described in this disclosure is a system for gene therapy using a chimeric vector made from an adenovirus genome and a heterologous gene that functionally replaces an adenovirus gene required for replication or assembly. Cytolytic viruses can be produced that target particular tissue types by virtue of having replication controlled by a specific transcription control element—such as the promoter for telomerase reverse transcriptase. These therapeutic viruses are believed to have an improved safety and efficacy profile compared with previously available systems.

### REFERENCE TO RELATED APPLICATIONS

This application claims priority to U.S. patent application Ser. No. 60/256,418, filed Dec. 18, 2001, pending. For purposes of prosecution in the U.S., the priority application and U.S. patent application Ser. No. 09/615,039 are hereby incorporated herein in their entirety.

### TECHNICAL FIELD

This invention relates generally to the fields of virology and gene therapy. More specifically, the disclosure provides chimeric viruses comprising a heterologous replication element in a adenovirus-based construct for cytolysis of specific target cells.

### BACKGROUND

Many forms of cancer are intractable to traditional courses of radiation or small molecule pharmaceuticals. Considerable interest has evolved in developing gene therapy vectors as therapeutic agents.

A broad variety of therapeutic genes are currently under investigation in preclinical and in clinical studies (reviewed by Walther et al., Mol. Biotechnol. 13:21, 1999). The candidate genes have different origins and mechanisms of action, such as cytokine genes, genes coding for immunostimulatory molecules/antigens, genes encoding prodrug-activating enzymes (suicide genes), genes that promote apoptosis, and tumor suppressor genes.

Vectors for delivering such genes can be based on viral and non-viral systems. For example, viral vectors can be based on herpes family viruses. U.S. Pat. No. 5,728,379 (Georgetown University) relates to replication competent HSV containing a transcriptional regulatory sequence operatively linked to an essential HSV gene. U.S. Pat. No. 5,997,859 and EP 702084 B1 (Chiron) pertain to replication-defective recombinant retrovirus, carrying a vector construct capable of preventing, inhibiting, stabilizing or reversing infections, cancer, or autoimmune disease. WO 99/08692 proposes the use of reovirus in treating cancer, particularly ras-mediated neoplasms.

Many proposed cancer therapeutic vectors are based on adenovirus. U.S. Pat. Nos. 5,631,236 and 6,096,718 (Baylor College of Medicine) cover a method of causing regression in a solid tumor, using a vector containing an HSV thymidine kinase (tk) gene, followed by administration of a prodrug such as ganciclovir. U.S. Pat. No. 6,096,718 (Baylor College of Medicine) relates to the use of a replication incompetent adenoviral vector, comprising an HSV tk gene under control of the α-lactalbumin promoter.

U.S. Pat. No. 5,801,029 and 5,846,945 (Onyx Pharmaceuticals) relate to adenovirus in which the E1b gene has been altered so as not to bind and inactivate tumor suppressor p53 or RB proteins expressed by the host. This prevents the virus from inactivating tumor suppression in normal cells, which means the virus cannot replicate. However, the virus will replicate and lyse cells that have shut off p53 or RB expression through oncogenic transformation.

U.S. Pat. No. 5,998,205 and WO 99/25860 (GTI/Novartis) pertain to a tissue-specific replication-conditional adenovirus, comprising a transcriptional regulatory sequence (such as the α-fetoprotein promoter) operably linked to an adenovirus early replication gene. U.S. Pat. No. 5,698,443 (Calydon) provides replication-conditional adenoviruses controlled by the PSA promoter. Alemany et al. (Cancer Gene Ther. 6:21, 1999) outline complementary adenoviral vectors for oncolysis. One vector contains cis replication elements and E1a under control of a tissue-specific promoter. The supplemental vector contains all other trans-acting adenovirus replication genes. Coinfection leads to controlled killing of hepatocarcinoma cells.

International Patent Publication WO 98/14593 (Geron) describes an adenovirus construct in which the tk gene is placed under control of the promoter for telomerase reverse transcriptase (TERT). This gene is expressed at high levels in cancer cells of any tissue type, and the vector renders cancer cell lines susceptible to toxic effects of ganciclovir. WO 00/46355 (Geron) describes an oncolytic virus having a genome in which a TERT promoter is linked to a genetic element essential for replication or assembly of the virus, wherein replication of the virus in a cancer cell leads to lysis of the cancer cell.

Koga et al. (Hu. Gene Ther. 11:1397, 2000) proposed a telomerase-specific gene therapy using the human TERT gene promoter linked to the apoptosis gene Caspase-8 (FLICE). Gu et al. (Cancer Res. 60:5359, 2000) reported a binary adenoviral system that induced Bax expression via the hTERT promoter. They found that it elicited tumor-specific apoptosis in vitro and suppressed tumor growth in nude mice.

There is a need to develop new constructs with improved safety and efficacy for use in cancer therapy.

### SUMMARY OF THE INVENTION

This disclosure provides a system for gene therapy of target tissues using chimeric particles made from an adenovirus genome and heterologous genetic elements. Cytolytic viruses can be produced that target particular tissue types, and are suitable for use in cancer therapy.

One embodiment of this invention is a viral vector having a genome comprising adenovirus replication genes, and at least one heterologous gene that functionally replaces an adenovirus gene required for replication or assembly. The vector can be cytolytic to the host cell upon replication. For example, the E1a gene (or subfunction thereof) can be replaced by a Y-box transactivator such as YB-1, the immediate early genes of cytomegalovirus such as IE1 or IE2, or the E6E7 oncogenes from papillomavirus.

Furthermore, chimeric vectors of this invention can be made replication-conditional by placing the heterologous gene or any other gene required for replication or assembly of the vector under control of a tissue or tumor specific transcriptional control element. Suitable control elements are illustrated elsewhere in this disclosure, exemplified by a promoter sequence for telomerase reverse transcriptase (TERT).

This disclosure teaches methods for selecting chimeric viruses of this invention by transducing a host cell with a virus lacking an adenovirus gene required for replication or assembly, but comprising a heterologous gene; and determining whether replicated virus is produced by the cell.

Another embodiment of this invention is a method for killing a cancer cell or a cell expressing TERT, comprising combining the cell with a cytopathic virus of this invention.

Other embodiments of the invention are medicaments and methods for treating a human or animal body for a condition targetable by a replication-conditional cytopathic virus—such as cancer, or any other abnormality associated with increased expression of TERT in affected cells.

A further embodiment of the invention is a cell line for producing an adenovirus vector that is replication-defective because it is missing a gene required for replication or assembly. The cell line comprises a non-adenovirus gene (such as a Y box transactivator or a CMV immediate early gene) that functionally replaces the missing gene (such as E1a), under control of a transcriptional control element that is heterologous to the non-adenovirus gene.

Other embodiments of the invention will be apparent from the description that follows.

### DRAWINGS

FIG. 1 is a series of three maps showing model adenovirus made conditionally replicative by placing the E1a replication under control of an hTERT promoter.

FIG. 2 is a half tone reproduction of cell lines photographed 7 days after infection with oncolytic virus. Top row: uninfected cells (negative control). Middle row: cells infected with oncolytic adenovirus, in which replication gene E1a is operably linked to the hTERT promoter. Bottom row: cells infected with adenovirus in which E1a is operably linked to the CMV promoter (positive control).

The cells tested were as follows: Top panels: BJ (foreskin fibroblast); IMR-90 (lung fibroblast); WI-38 (lung fibroblast); cells of non-malignant origin. Bottom panels: DAOY (medulloblastoma); HeLa (cervical carcinoma); HT1080 (fibrosarcoma). The results show that the hTERT-regulated oncolytic virus (AdhTERTpE1a) specifically lyses cancer cells, in preference to cell lines that don't express telomerase reverse transcriptase at a substantial level. This is in contrast to oncolytic virus regulated by a constitutive promoter like CMV promoter (AdCMVpE1), which lyses cells non-specifically.

FIG. 3 is a graph showing the effect of an adenovirus driven by the hTERT promoter on osteosarcoma tumors in a mouse model. The group labeled “Ad5Emp” is the vector control, with tumors growing to 400 mm³ within a month of injection. The groups labeled “Onco2” were injected intratumorally with the replication-conditional vector on days 11-15, and showed considerable reduction in tumor growth rate.

### DETAILED DESCRIPTION

A promising therapeutic approach to cancer is to target tumors with a virus that replicates specifically in cancer cells, destroying them in the process. Replication conditional adenovirus and herpes virus constructs have been made and are currently in clinical trials.

However, using adenovirus derivatives for human therapy has significant risks—illustrated by the recent tragedy in the ornithine transcarbamylase gene therapy trial conducted at the University of Pennsylvania (M. Balter, Science 288:951, 2000). Furthermore, replication competent adenovirus can transmit from cell to cell, which can increase efficacy in cancer cell killing, but also increases the degree of caution exercised during development of the technology.

It is a hypothesis of this invention that safety of replication-competent adenovirus can be improved by substituting one or more of the genes required for replication or assembly with a gene from another source. This may lessen the risk of recombination with any coinfecting naturally occurring virus. It may also lessen the collateral effects that adenovirus genes have on the cells they infect.

For example, the E1a gene products are believed to have the potential for a wide range of biological activities—activation and repression of transcription, induction of DNA synthesis, mitosis, apoptosis, suppression or induction of differentiation, cell immortalization, various cytotoxic activities, and susceptibility of the cell to TNF and a host cellular immune response. In cooperation with the E1b gene products or activated ras, E1a can help transform the infected cell into a malignant phenotype.

This invention proposes replication-competent constructs in which E1a is replaced with a Y box transactivator of human origin. In this way, the undesirable transforming activity of E1a is eliminated—but the replicative capacity (and hence the therapeutic benefit) of the construct remains intact.

The chimeric viruses of this invention can be made to replicate preferentially in cancer cells by placing at least one gene under control of a promoter that is up-regulated in cancer cells. Of particular interest is the promoter for telomerase reverse transcriptase (TERT), which is expressed in almost all cancer cell lines. Telomerase allows the cancer cells to grow beyond the maximum number of doublings permitted a normal cell (the Hayflick limit). In contrast, terminally differentiated cells in the body usually do not express telomerase, and the few cells that do contain telomerase have only about 2-5 copies per cell.

This means that viruses that replicate under control of the TERT promoter are exquisitely specific for cancer cells—they should be able to target cancer cells of essentially any tissue, but will not be able to replicate in adjacent non-malignant cells in the same tissue.

Further advantages of the chimeric vectors of this invention are described in the sections that follow.

Definitions

The term “polynucleotide” refers to a polymeric form of nucleotides of any length. Included are genes and gene fragments, mRNA, cDNA, recombinant polynucleotides, branched polynucleotides, plasmids, viral and non-viral vectors and particles, nucleic acid probes, amplification primers, and their chemical equivalents. As used in this disclosure, the term polynucleotide refers interchangeably to double- and single-stranded molecules. Unless otherwise specified, any embodiment of the invention that is a polynucleotide encompasses both a double-stranded form, and each of the two complementary single-stranded forms known or predicted to make up the double-stranded form.

A cell is said to be “genetically altered”, “transfected”, or “genetically transformed” when a polynucleotide has been transferred into the cell by any suitable means of artificial manipulation, or where the cell is a progeny of the originally altered cell that has inherited the polynucleotide.

A “control element” or “control sequence” is a nucleotide sequence that contributes to the functional regulation of a polynucleotide, such as replication, duplication, transcription, splicing, translation, or degradation of a polynucleotide. Transcriptional control elements include promoters, enhancers, and repressors.

Particular gene sequences referred to as promoters, like the “TERT promoter”, or the “PSA promoter”, are polynucleotide sequences derived from the gene referred to that promote transcription of an operatively linked gene expression product. It is recognized that various portions of the upstream and intron untranslated gene sequence may in some instances contribute to promoter activity, and that all or any subset of these portions may be present in the genetically engineered construct referred to. The promoter may be based on the gene sequence of any species having the gene, unless explicitly restricted, and may incorporate any additions, substitutions or deletions desirable, as long as the ability to promote transcription in the target tissue is maintained. Genetic constructs designed for treatment of humans may comprise a segment that is at least 90% identical to a promoter sequence of a human gene. A particular transcription control sequence can be tested for activity and specificity, for example, by operatively linking to a reporter gene (Example 1).

When comparison is made between polynucleotides for degree of identity, it is implicitly understood that complementary strands are easily generated, and the sense or antisense strand is selected or predicted that maximizes the degree of identity between the polynucleotides being compared. Percentage of sequence identity is calculated by first aligning the polynucleotide being examined with the reference counterpart, and then counting the number of residues shared between the sequences being compared as a percentage of the region under examination, without penalty for the presence of obvious insertions or deletions.

Genetic elements are said to be “operatively linked” if they are in a structural relationship permitting them to operate in a manner according to their expected function. For instance, if a promoter initiates transcription of a coding sequence, the coding sequence can be referred to as operatively linked to (or under control of) the promoter. There may be intervening sequence between the promoter and coding region so long as this functional relationship is maintained.

In the context of encoding sequences, promoters, and other gene elements, the term “heterologous” indicates that the element is derived from a genotypically distinct entity from that of the rest of the entity to which it is being compared. For example, a promoter or gene introduced by genetic engineering techniques into a context in which it does not occur in nature is said to be a heterologous polynucleotide. A heterologous gene or heterologous promoter in an adenovirus vector is a genetic element derived from another virus, a genetic element derived from a prokaryote or eukaryote, or an artificial sequence unrelated to adenovirus. An “endogenous” genetic element is an element that is in the same place in the chromosome or viral genome where it occurs in nature, although other gene elements may be artificially introduced into a neighboring position.

A “replication-conditional” virus comprises a gene essential for replication or assembly of the virus that is preferentially transcribed in cells of a certain type, compared with other cells of the same species. Viruses can be made replication-conditional by placing a gene required for replication or assembly under control of a transcriptional control element that activates or derepresses transcription in certain cell types, compared with others. Exemplary transcriptional control elements are listed later in this disclosure.

A “cytolytic virus” is a virus that lyses or kills a host cell by replicating in the cell, thereby causing the cell to rupture. An “oncolytic virus” is a cytolytic virus that is replication-conditional for cancer cells (and possibly other cell types). It is understood that such viruses are not confined to use with cancer cells, and can be used in vitro or in vivo for any desirable purpose.

Unless otherwise required, the viral vectors, techniques, methods, and other embodiments of this invention can be practiced using adenovirus capable of infecting any mammalian cell, including but not limited to human adenovirus. The description that follows is modeled on adenovirus type 2 or type 5, and can be readily adapted to other adenovirus types by substituting the genetic homologs wherever necessary or desired.

General Techniques

Methods in molecular genetics and genetic engineering are described generally in the current editions of Molecular Cloning: A Laboratory Manual, (Sambrook et al.); Oligonucleotide Synthesis (M. J. Gait, ed.); Animal Cell Culture (R. I. Freshney, ed.); Gene Transfer Vectors for Mammalian Cells (Miller & Calos, eds.); Current Protocols in Molecular Biology and Short Protocols in Molecular Biology, 3rd Edition (F. M. Ausubel et al., eds.); and Recombinant DNA Methodology (R. Wu ed., Academic Press).

For general principles in vector construction, the reader is referred to Viral Vectors: Basic Science and Gene Therapy (Arrequi & Garcia-Carranca, eds., Eaton Pub. Co., 2000). Reagents, cloning vectors, and kits for genetic manipulation referred to in this disclosure are available from commercial vendors such as BioRad, Stratagene, Invitrogen, and ClonTech.

For a description of the molecular biology of cancer, the reader is referred to Principles of Molecular Oncology (M. H. Bronchud et al. eds., Humana Press, 2000); The Biological Basis of Cancer (R. G. McKinnel et al. eds., Cambridge University Press, 1998); and Molecular Genetics of Cancer (J. K. Cowell ed., Bios Scientific Publishers, 1999).

General techniques for the development, testing, and administration of biomolecular chemotherapeutics are provided in Gene Therapy of Cancer, Adv. Exp. Med. Biol. vol. 451 (P. Walden ed., Plenum Publishing Corp., 1998); Cancer Gene Therapy, Adv. Exp. Med. Biol. vol. 465(N. A. Habib ed., Kluwer Academic Pub, 2000); and Gene Therapy of Cancer: Methods and Protocols, Meth. Mol. Med. vol. 35 (W. Walther & U. Stein eds., Humana Press, 2000).

Construction of Chimeric Virus

The vectors of this invention are made up principally of an adenovirus genome, in which at least one gene essential for replication or assembly of the virus is replaced with a heterologous (non-adenovirus) gene.

Adenoviruses are nonenveloped, regular icosahedrons. Early in viral replication, the E1a gene is translated, producing a pleiotropic protein that is a transactivator for other early genes (E1b, E2, E3, and E4). The E1b gene product acts on the host nucleus to alter function of the host cell such that processing and transport are shifted to the late genes, which cause packaging of the virus into its capsid and release from the cell. Adenovirus DNA includes inverted terminal repeat sequences (ITRs) of ˜100-150 base pairs, which enables single strands of viral DNA to circularize by base pairing of the termini. There is also a packaging signal of a few hundred base pairs, and both the ITRs and the packaging signal are required for packaging the replicated genome into an adenovirus particle.

Any gene (or genetic element) can be replaced in the vector, providing the vector can be packaged at a sufficient rate. Where the vector is a cytolytic virus, the vector is replication-competent, which means it can replicate in a suitable host cell or tissue in the absence of adenovirus genes not present in the virus itself.

Of particular interest for functional replacement are the adenovirus early genes, especially E1a, E1b, E2 and E4. E1a can be functionally replaced by a select group of transactivators also capable of promoting transcription of E1b, E2, and E4, that typically also modulate endogenous gene expression in the host cell. Since E1a is pleiotropic, the invention will be understood to include embodiments in which one of the functional subelements of E1a is deleted or suppressed, and substituted with a heterologous gene. Similarly, E2 can be replaced by an encoding region for one or more proteins that mimic the function of the E2 gene products: a single-stranded DNA binding protein, a DNA polymerase, and a terminal protein.

Candidate transactivators to replace E1a include viral transactivator genes from other viruses, such as members of the herpes simplex virus family, and SV40. Of particular interest are the immediate early genes from cytomegaloviruses (CMV) that are cytopathic for humans or other vertebrates—including the genes known as IE1 and IE2 (SEQ. ID NO:4). Immediate early genes function to regulate viral and cellular gene expression during the course of CMV replication. The major IE region of the CMV genome is believed to activate viral genes and represses genes of the host cell. The molecular biology of CMV is reviewed by Emery et al. (Int. J. Exp. Pathol. 71:905, 1990) . Function and regulation of CMV IE genes are reviewed by Stenberg (Intervirology 39:343; 1996); Meier et al. (Intervirology 39:331; 1996); Spector (Intervirology 39:361; 1996); Spector et al. (Virology 151:329; 1986); and Tevethia et al. (Virology 161:276, 1987).

Other candidate transactivators include transactivator genes from higher eukaryotes, especially humans. Of particular interest is the family of Y box transactivators, including YB-1 (SEQ. ID NOs:3 & 4). The specificity of the Y box transactivators is reviewed by R. Mantovani (Nucl. Acids Res. 26:1135, 1998 and Swamynathan et al. (FASEB J. 12:515, 1998). Didier et al. (Proc. Natl. Acad. Sci. USA 85:7433, 1988) investigated the cis-acting elements that regulate HLA Class II gene expression through the Y box (containing an inverted CCAAT box). By probing a phage λgt11 library with double-stranded oligonucleotides, they isolated cDNA for YB-1. It encodes a 35,414 kDa protein that has an absolute requirement for the CCAAT box and relative specificity for the Y box. There is an inverse correlation of YB-1 and HLA-DR β chain expression. YB-1 interacts with proliferating cell nuclear antigen (Ise et al., Cancer Res. 49:342, 1999), and may translocate to the nucleus by a protein kinase C mediated signal transduction pathway (FEBS Lett. 417, 390, 1997). YB-1 expression can be modulated by antisense compounds (U.S. Pat. No. 6,140,126). A further example is the E6 and E7 oncoprotein genes of human papillomavirus (Farthing et al., Trends Microbiol. 2:170, 1994; Jones et al., Semin. Cancer Biol. 7:327, 1996). E6E7 can mediate replication of adenovirus that has been mutated to delete the CR1/CR2 domains of E1a.

The manipulation and molecular biology of adenovirus vectors is generally described in Adenovirus Methods and Protocols, Methods in Molecular Medicine Vol. 21, (W. S. M. Wold ed., Humana Press, 1998), and the current edition of Field's Virology (Lippincott Williams & Wilkins, 1996). Other publications of interest include Danthinne et al., Gene Ther. 7:1707, 2000, Bilbao et al., Adv. Exp. Med. Biol. 451:365, 1998, and U.S. Pat. Nos. 5,631,236 (Baylor College of Medicine), 5,670,488 (Genzyme), 5,698,443 (Calydon), 5,712,136 (GenVec), 5,880,102 (Duke University), 5,994,128 (IntroGene), 6,040,174 (Transgene), and 6,096,718 (Gene Targeting Corp).

Replacing an adenovirus gene with a non-adenovirus functional equivalent can proceed by any genetic engineering strategy appropriate for vectors of this kind. Typically, a bacterial plasmid comprising the appropriate region of the adenovirus genome is engineered to replace an adenovirus gene required for replication or assembly with the substitute non-adenovirus gene. The plasmid is then used as a shuttle vector, and combined with a second plasmid containing the complementary portion of the adenovirus genome, permitting homologous recombination to occur by virtue of shared adenovirus sequences in the two plasmids. The recombination can be done directly in a suitable mammalian host (such as 293 cells), or else in yeast YAC clones or E. coli, subsequently transfecting the completed adenovirus genome into mammalian host cells for replication and encapsulation.

Testing and validation of chimeric adenovirus vectors is explained below.

Specific Transcriptional Control Elements

This invention includes replication-conditional viruses, wherein at least one gene required for replication of the vector is placed under control of a heterologous transcription control element. In chimeric viruses, this may be one of the endogenous replication or packaging genes, or a heterologous gene that replaces the function of an endogenous gene. The control element is selected with a view to the protein expression patterns in a target cell type (such as a malignant cell), compared with other cells that will be exposed to the vector in its normal use.

Many tumor-specific transcriptional control elements can be used in this invention. These control elements cause elevated transcription of the encoding sequence they are linked to in tumor cells of a variety of different types. Examples are promoters that control telomerase reverse transcriptase (TERT), carcinoembryonic antigen (CEA), hypoxia-responsive element (HRE), autocrine motility factor receptor (Grp78), L-plastin, and hexokinase II.

The promoter for TERT is an exemplary tumor-specific promoter. Sequence of the human TERT gene (including upstream promoter sequence) is provided below. International Patent Publication WO 00/46355 (Morin et al., Geron Corporation) describes and illustrates the construction and use of oncolytic virus (for example, adenovirus or HSV) that conditionally replicate under control of the human TERT promoter. The reader is also referred to U.K. Patent GB 2321642 B (Cech et al., Geron Corporation and U. Colorado), International Patent Publication WO 99/33998 (Hagen et al., Bayer Aktiengesellschaft), and Horikawa et al. (Cancer Res., 59:826, 1999).

A lambda phage clone designated λGΦ5, containing ˜13,500 bases upstream from the hTERT encoding sequence, is available from the ATCC under Accession No. 98505. Suitable TERT promoter may comprise a sequence of 25, 50, 100, or 200 consecutive nucleotides that is 80%, 90%, or 100% identical (or can hybridize under stringent conditions) to a sequence contained in SEQ. ID NO:1. Example 1 illustrates the testing and use of TERT promoter sequences in vector expression systems. Those skilled in the art will appreciate that promoter sequences not contained in λGΦ5 but homologous and capable of promoting preferential expression in cancer cells can be used with similar effect—including but not limited to TERT promoters from other species, artificial TERT promoter sequences, and chimeric sequences. The mouse TERT sequence is provided in WO 99/27113 (Morin et al., Geron Corporation).

As an alternative, a transcriptional control element can be used that is tissue-specific. Constructs of this kind will cause preferential expression of the operatively linked encoding region if the level of expression of the endogenous gene is higher in tumor cells than in non-malignant tissue of the same type. They are also useful to treat tumors that have metastasized away from the primary site. Examples are promoters that control transcription of albumin (liver-specific), a-fetoprotein (AFP, liver-specific), prostate-specific antigen (PSA, prostate-specific), mitochondrial creatine kinase (MCK, muscle-specific), myelin basic protein (MBP, oligodendrocyte-specific), glial fibrillary acidic protein (GFAP, glial cell specific), and neuron-specific enolase (NSE, neuron-specific). See U.S. Pat. No. 5,871,726 (Calydon), WO 98/39466 (Calydon), U.S. Pat. No. 5,998,205, and WO 99/25860 (Genetic Therapy Inc.).

Additional promoters suitable for use in this invention can be taken from other genes that are preferentially expressed in tumor cells. Such genes can be identified, for example, by differential display and comparative genomic hybridization: see U.S. Pat. Nos. 5,759,776 and 5,776,683. Alternatively, microarray analysis can be performed by comparing mRNA preparations from cancer cells and a matched non-malignant control. Preferably, the level of expression of the effector gene will be at least 5-fold or even 25-fold higher in the undifferentiated cells relative to the differentiated cells. Having identified transcriptional control elements of interest, specificity can be tested in a reporter construct where the control element is used to control transcription of a reporter gene (Example 1).

Optionally, the chimeric virus can comprise both a first heterologous transcription control element that provides specificity of replication, and a second heterologous transcription control element that accentuates the replication rate. For example, a gene required for replication of the virus can be operatively linked to both the hTERT promoter, and to a non-specific constitutive enhancer, such as the CMV enhancer (U.S. Pat. No. 5,849,522) or the SV40 or polyomavirus enhancer (U.S. Pat. No. 4,963,481).

In some embodiments of the invention, the adenovirus vector further comprises an effector gene that contributes to killing of the infected cell by another mechanism. Suitable effector genes encode a peptide toxin (such as ricin or diphtheria toxin), or a gene product that induces or mediate apoptosis (such as Caspase-8 or Bax). Other suitable effectors encode polypeptides having activity that is not directly toxic to a cell, but renders the cell sensitive to an otherwise nontoxic compound. Exemplary is thymidine kinase, which converts the anti-herpetic agent ganciclovir to a toxic product that interferes with DNA replication in proliferating cells (U.S. Pat. No. 5,631,236 and EP 657541 A1).

Adenovirus Vector Producing Cell Lines

This invention also provides host cells that can be used to produce replication-defective adenovirus vectors by replacing the function of an adenovirus gene required for replication or assembly with a non-adenovirus gene engineered into the host cell under control of a heterologous promoter.

These producing cell lines provide an advantage over previously used lines. For example, 293 cells are effective for packaging because they contain genes from adenovirus type 5 that complement the deficiency in the virus being replicated. However, there is still a small amount of replication-competent virus produced under some conditions, which for transient gene therapy is undesirable. It is a hypothesis of this invention that the proportion of replication-competent virus will be lower in a producing cell in which the genes complementing the missing adenovirus function are derived from a different virus or from a prokaryotic or eukaryotic cell. Because no genetic material is shared with the adenovirus vector being produced, there will be less opportunity for a recombination event that creates a replication competent particle.

The principles for replacing the adenovirus gene in the producing cell line run parallel to those already described for the cytolytic viruses described above. For example, vector particles deficient in the E1a gene can be produced in cells that express either a Y box protein such as YB-1, or a CMV immediate early gene such as IE1 or IE2.

The replacing gene is put under control of a heterologous transcription control element that promotes transcription of the gene in the selected host cell in either a constitutive or inducible fashion. In one example, the replacement gene is inserted by heterologous recombination behind a promoter endogenous and active in the host cell. In another example, a vector is constructed in which the replacement gene is operatively linked to a promoter from another source—such as the SV40 promoter, or the promoter for phosphoglycerate kinase (PGK). The vector is then transfected into a suitable host cell, from which the genetically altered line is cloned by limiting dilution. The transcriptional control element is chosen so that the cell produces a useful quantity of adenovirus particles at a reasonable rate. Optimally, at least ˜100 or ˜1000 particles will be produced per cell.

Replication defective vectors made by these producing cell lines can be used for any desirable purpose in vivo or in vitro. For example, adenovirus vectors can be used to provide transient expression of a heterologous therapeutic gene to correct a congenital defect, to introduce proinflammatory or anti-inflammatory activity, or to enhance telomerase function. Adenovirus vectors can also be used to deliver heterologous effector genes that induce killing of the transduced cell, such as those exemplified in the previous section.

Formulation and Testing of Cvtolvtic Virus

Whether a particular non-adenovirus gene can adequately replace the function of an adenovirus gene required for assembly or replication can be determined by several different empirical screening methods.

In one method, a putative vector producing cell line as described in the preceding section is constructed by genetically altering the cell to express the candidate replacing gene under control of a promoter that is heterologous to the gene. The cell is also introduced with a viral vector, plasmid, or plasmid combination, comprising all the genetic elements required for replication and assembly of adenovirus, except the gene being replaced.

The producer cell line is then monitored for production of viral particles—for example, by measuring DNA synthesis by [³H]thymidine incorporation, immunohistochemistry or RT-PCR for a late gene product (such as hexon), by counting virions (using electron microscopy or plaque assay), or by killing of the host cells (TCID₅₀).

Another screening method involves constructing a putative cytolytic replication-competent virus, comprising a genome in which the candidate gene replaces a required adenovirus gene. The ability of the heterologous gene to functionally substitute can then be tested by comparing the replicative or lytic property of the new construct with that of a virus containing the endogenous gene. Example 2 illustrates the evaluation of oncolytic virus by testing the effect on a panel of cancer cell lines, and comparing with the effect on other cell lines.

Further validation of a virus of this invention for the treatment of a particular condition can be tested in a suitable animal model. For example, efficacy for treating cancer can be determined using mice injected with a representative human cancer cell line, such as a glioma or osteosarcoma. After solid tumors have developed of a sizeable diameter, the mice are injected intravenously or intratumorally with the chimeric vector, for example, in a dose range of 10⁶ to 10⁹ pfu, and then monitored for reduced tumor growth rate and increased survival (Example 5).

Dosage and formulation of medicaments intended for human therapy are based on the animal model experiments. For general guidance on formulation and testing of medicament formulations for human administration, the reader is referred to Biopharmaceutical Drug Design and Development (S. Wu-Pong et al. eds, Humana Press 1999); Biopharmaceuticals: Biochemistry and Biotechnology (G. Walsh, John Wiley & Sons, 1998); and the most current edition of Remington: The Science and Practice of Pharmacy (A. Gennaro, Lippincott, Williams & Wilkins). Pharmaceutical compositions of this invention may be packaged in a container with written instructions for use of the cells in human therapy, and the treatment of cancer.

The examples that follow are provided by way of further illustration, and are not meant to limit the claimed invention.

### EXAMPLES

### Example 1

Preparation of Vectors Controlling Transcription in Cells Expressing Telomerase Reverse Transcriptase

The lambda clone designated λGΦ5 containing the hTERT promoter is deposited with the American Type Culture Collection (ATCC), 10801 University Blvd., Manassas, VI 20110 U.S.A., under Accession No. 98505. λGΦ5 contains a 15.3 kbp insert including approximately 13,500 bases upstream from the hTERT coding sequence.

A Not1 fragment containing the hTERT promoter sequences was subcloned into the Not1 site of pUC derived plasmid, which was designated pGRN142. A subclone (plasmid pGRN140) containing a 9 kb Ncol fragment (with hTERT gene sequence and about 4 to 5 kb of lambda vector sequence) was partially sequenced to determine the orientation of the insert. pGRN140 was digested using SaII to remove lambda vector sequences, the resulting plasmid (with removed lambda sequences) designated pGRN144. The pGRN1 44 insert was then sequenced.

SEQ. ID NO:1 is a listing of the sequence data obtained. Nucleotides 1-43 and 15376-15418 are plasmid sequence. Thus, the genomic insert begins at residue 44 and ends at residue 15375. The beginning of the cloned cDNA fragment corresponds to residue 13490. There are Alu sequence elements located ˜1700 base pairs upstream. The sequence of the hTERT insert of pGRN142 can now be obtained from GenBank (http://www.ncbi.nlm.nih.gov/) under Accession PGRN142.INS AF121948. Numbering of hTERT residues for plasmids in the following description begins from the translation initiation codon, according to standard practice in the field. The hTERT ATG codon (the translation initiation site) begins at residue 13545 of SEQ. ID NO:1. Thus, position −1, the first upstream residue, corresponds to nucleotide 13544 in SEQ. ID NO:1.

Expression studies were conducted with reporter constructs comprising various hTERT upstream and intron sequences. A BgIII-Eco47III fragment from pGRN144 (described above) was digested and cloned into the BgIII-Nrul site of pSEAP2Basic (ClonTech, San Diego, Calif.) to produce plasmid designated pGRN148. A second reporter-promoter, plasmid pGRN150 was made by inserting the BgIII-Fspl fragment from pGRN144 into the BgIII-Nrul sites of pSEAP2. Plasmid pGRN173 was constructed by using the EcoRV-Stul (from +445 to −2482) fragment from pGRN144. This makes a promoter reporter plasmid that contains the promoter region of hTERT from approximately 2.5 kb upstream from the start of the hTERT open reading frame to just after the first intron within the coding region, with the initiating Met codon of the hTERT open reading frame changed to Leu. Plasmid pGRN175 was made by APA1(Klenow blunt)-SRF1 digestion and religation of pGRN150 to delete most of the Genomic sequence upstream of hTERT. This makes a promoter/reporter plasmid that uses 204 nucleotides of hTERT upstream sequences (from position −36 to −117). Plasmid pGRN176 was made by PML1-SRF1 religation of pGRN150 to delete most of the hTERT upstream sequences. This makes a promoter/reporter plasmid that uses 204 nucleotides of hTERT upstream sequences (from position −36 to −239).

Levels of secreted placental alkaline phosphatase (SEAP) activity were detected using the chemiluminescent substrate CSPDTM (ClonTech). SEAP activity detected in the culture medium was found to be directly proportional to changes in intracellular concentrations of SEAP mRNA. The pGRN148 and pGRN150 plasmids (hTERT promoter-reporter) and the pSEAP2 plasmid (positive control, containing the SV40 early promoter and enhancer) were transfected into test cell lines. pGRN148 and pGRN150 constructs drove SEAP expression as efficiently as the pSEAP2 in immortal (tumor-derived) cell lines. Only the pSEAP2 control gave detectable activity in mortal cells.

The ability of the hTERT promoter to specifically drive the expression of the thymidine kinase (tk) gene in tumor cells was tested using a variety of constructs: One construct, designated pGRN266, contains an EcoRI-Fsel PCR fragment with the tk gene cloned into the EcoRI-Fsel sites of pGRN263. pGRN263, containing approximately 2.5 kb of hTERT promoter sequence, is similar to pGRN150, but contains a neomycin gene as selection marker. pGRN267 contains an EcoRI-Fsel PCR fragment with the tk gene cloned into the EcoRI-Fsel sites of pGRN264. pGRN264, containing approximately 210 bp of hTERT promoter sequence, is similar to pGRN176, but contains a neomycin gene as selection marker. pGRN268 contains an EcoRI-Xbal PCR fragment with the tk gene cloned into the EcoRI-Xbal (unmethylated) sites of pGRN265. pGRN265, containing approximately 90 bp of hTERT promoter sequence, is similar to pGRN175, but contains a neomycin gene as selection marker.

These hTERT promoter/tk constructs, pGRN266, pGRN267 and pGRN268, were re-introduced into mammalian cells and tk/+ stable clones (and/or mass populations) were selected. Ganciclovir treatment in vitro of the tk/+ cells resulted in selective destruction of all tumor lines tested, including 143B, 293, HT1080, Bxpc-3′, DAOY and NIH3T3. Ganciclovir treatment had no effect on normal BJ cells.

### Example 2

Killing Cancer Cells Using Vectors Controlled by the TERT Promoter

A replication-conditional adenovirus was constructed by placing a gene involved in viral replication under control of the hTERT promoter, which should activate transcription in telomerase-expressing cancer cells. The viral construct comprised the Inverted Terminal Repeat (ITR) from adenovirus Ad2; followed by the hTERT medium-length promoter (phTERT176) operably linked to the adenovirus E1a region; followed by the rest of the adenovirus deleted for the E3 region (ΔE3). As a positive control, a similar construct was made in which E1a was placed under control of the CMV promoter, which should activate transcription in any cell.

Reagents were obtained as follows. pBR322, restriction enzymes: NEB, Beverly, Mass. Adenovirus Type 2 (Ad2), tissue culture reagents: Gibco/BRL, Grand Island, N.Y. Profection™ Mammalian Transfection Systems: Promega, Madison, Wis. Tumor and Normal Cell lines: ATCC, Manassas, Va., except BJ line, which was obtained from J. Smith, U. of Texas Southwestern Medical Center.

Briefly, a pBR322-based plasmid was constructed which contains the Adenovirus Type 2 genome with deletions from 356-548nt (E1a promoter region) and 27971-30937nt (E3). A multiple cloning region was inserted at the point of deletion of the E1a promoter, and hTERT promoter (−239 to −36nt) or CMV promoter (−524 to −9nt) was subsequently cloned. Numbering of the CMV sequence is in accordance with Akrigg et al., Virus Res. 2:107, 1985. Numbering of the Ad2 sequence is in accordance with “DNA Tumor Viruses: Molecular Biology of Tumor Viruses”, J. Tooze ed., Cold Spring Harbor Laboratory, NY.

These plasmid DNAs were digested with SnaBI to liberate ITRs, then phenol-chloroform extracted, precipitated and transfected into 293A cells for propagation of the virus. Several rounds of plaque purifications were performed using A549 cells, and a final isolate was expanded on these same cells. Viruses were titered by plaque assay on 293A cells, and tested for the presence of 5′ wild type Ad sequences by PCR. DNA was isolated from viruses by HIRT extraction.

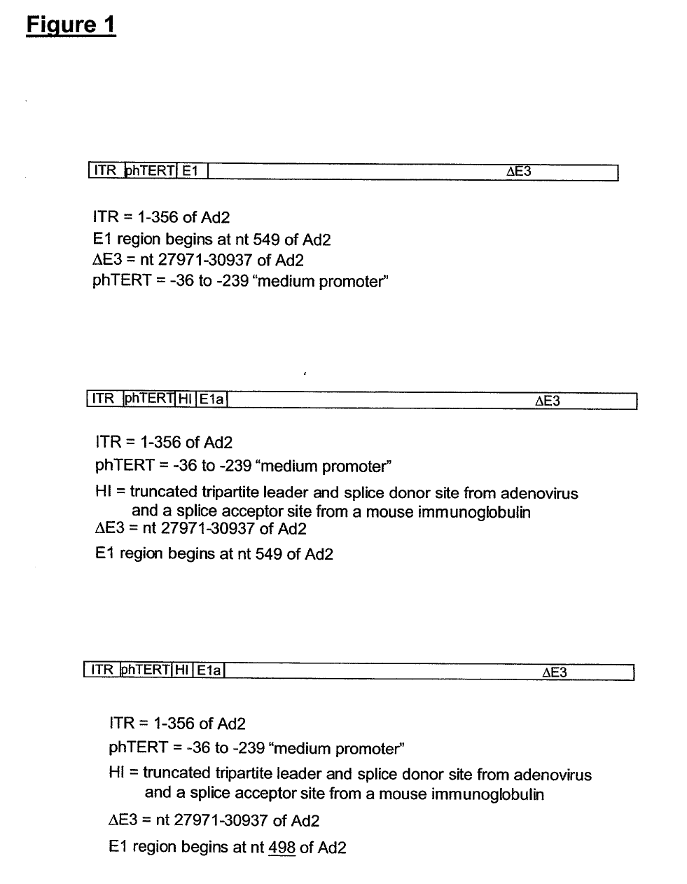

FIG. 1 is a map of model oncolytic adenovirus constructs. The vector shown at the top comprises the Inverted Terminal Repeat (ITR) from the adenovirus (Ad2); followed by the hTERT medium-length promoter (phTERT1 76) operably linked to the adenovirus E1a region; followed by the rest of the adenovirus deleted for the E3 region (ΔE3). Shown underneath are some modified constructs. The middle construct comprises an additional sequence in between the hTERT promoter and the E1a region. The HI sequence is an artificial intron engineered from adenovirus and immunoglobulin intron splice donor and acceptor sequences. It is thought that placing an intron in the hTERT promoter adenovirus replication gene cassette will promote processing and transport of heteronuclear RNA, thereby facilitating formation of the replicated viral particles. The third adenovirus construct is similar, except that the E1a region used is longer at the 5′ end by 51 nucleotides. It is thought that this may also promote more efficient conditional replication of the oncolytic virus.

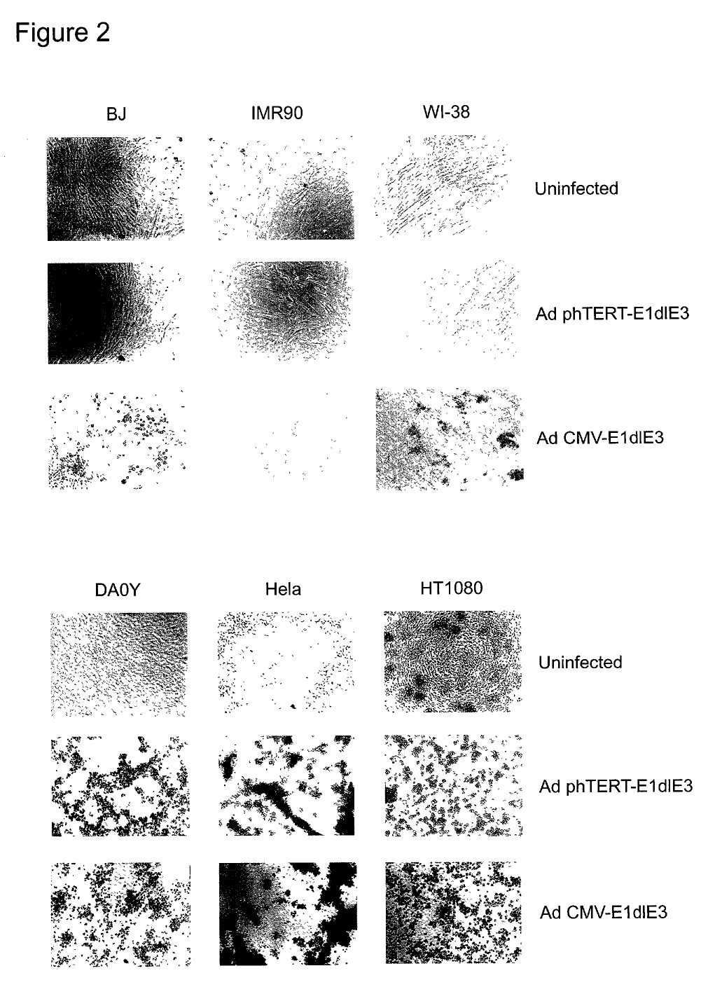

FIG. 2 shows the effect of these viruses on normal and cancer-derived cell lines. Each cell line was plated and infected at an MOI=20, ˜24h post plating. The cells were then cultured over a period of 17-48 days, and fed every fourth day. The pictures shown in the Figure were taken 7 days after infection. The top row of each section shows the results of cells that were not virally infected (negative control). The middle row shows the results of cells infected with oncolytic adenovirus, in which replication gene E1a is operably linked to the hTERT promoter. The bottom row of each section shows the results of cells infected with adenovirus in which E1a is operably linked to the CMV promoter (positive control). Results are summarized in Table 1.

TABLE 1
Effect of Oncolytic Virus on Cancerous and Non-cancerous Cells

| | | | Uninfected | Lysis by | Lysis by |
|---|---|---|---|---|---|
| | | | cell | phTERT- | pCMV- |
| Cell Line | Origin | Culture Conditions | Lysis | E1ΔE3 | E1ΔE3 |
| BJ | foreskin fibroblast | 90% DMEM/M199 + | NO | NO | YES |
| | | 10% FBS | | | |
| IMR | lung fibroblast | 90% DMEM/M199 + | NO | NO | YES |
| | | 10% FBS | | | |
| WI-38 | lung fibroblast | 90% DMEM/M199 + | NO | NO | YES |
| | | 10% FBS + | | | |
| | | 5 μg mL gentamicin | | | |
| A549 | lung carcinoma | 90% RPMI + | NO | YES | YES |
| | | 10% FBS | | | |
| AsPC-1 | adenocarcinoma, | 90% RPMI + | NO | YES | YES |
| | pancreas | 10% FBS | | | |
| BxPC-3 | adenocarcinoma, | 90% EMEM + | NO | YES | YES |
| | pancreas | 10% FBS | | | |
| DAOY | medulloblastoma | 90% EMEM + | NO | YES | YES |
| | | 10% FBS | | | |
| HeLa: | cervical | 90% EMEM + | NO | YES | YES |
| | carcinoma | 10% FBS | | | |
| HT1080 | fibrosarcoma | 90% EMEM + | NO | YES | YES |
| | | 10% FBS | | | |

All cell lines tested were efficiently lysed by AdCMV-E1dIE3 by day 17 post-infection. All tumor lines were lysed by AdphTERT-E1dIE3 in a similar, but slightly delayed period, while normal lines showed no signs of cytopathic effect and remained healthy out to 6 weeks post-infection.

The results demonstrate that an oncolytic virus can be constructed by placing a genetic element essential for replication of the virus under control of an hTERT promoter. Replication and lysis occurs in cancer cells, but not in differentiated non-malignant cells.

### Example 3

Substitution for E1a in a Cytolytic Oncovirus

The telomerase promoter cassette from the plasmid used in Example 2 (pBRAd2ITR/TB/phTERT/5′E1 a) containing the hTERT promoter (position −239 to −36) is amplified by PCR with 5′- and 3′-sequence-specific PCR primers containing a Kpnl linker site with a short 5′ heterologous flanking buffer sequence to maximize cutting efficiency inserted at the 5′ end of each primer. The PCR product is gel purified and cloned into the Kpnl site of the pShuttle™ transfer vector (Qbiogene, 2251 Rutherford Road, Carlsbad, Calif. 92008, part number AES1020, pShuttle™ vector). The resulting vector is verified for the correct insert by restriction mapping and sequencing and is designated pShuttle-phTERT.

Candidate E1a replacement genes are amplified by PCR with 5′- and 3′- PCR primers specific for the cDNA ends of the gene of interest with the addition at the 5′ end of each primer of bases 350 to 411 of the multiple cloning region from pShuttle. The resulting fragment is gel-purified and digested with EcoRV. DNA from the pShuttle-phTERT vector is also digested with EcoRV to linearize the plasmid and is then gel-purified and prepared for cloning. The EcoRV-digested fragment containing the candidate E1a replacement gene is then cloned into the EcoRV site of pShuttle-phTERT and transformed into competent E. coli DH5α cells. The resulting transformants are colony-purified and the candidate clones are screened for the insert by restriction digestion to ensure that the insert is in the appropriate orientation.

The shuttle vector with the verified insert is recombined in a bacterial homologous recombination system with an adenoviral backbone plasmid containing the remainder of the adenoviral genome up to and including the right ITR, to generate a full-length adenoviral construct. The resulting plasmid is linearized with Pmel and co-transformed into E. coli strain BJ5183 together with the E1-deleted adenovirus pAdEasy-1, the viral DNA plasmid (Qbiogene, 2251 Rutherford Road, Carlsbad, Calif. 92008, # AES1010 pAdEasy-1). Recombinants are selected with kanamycin and screened by restriction enzyme analysis. The recombinant adenoviral construct is then cleaved with Pac I to expose its ITRs, and transfected into 293 cells to produce viral particles.

The resulting adenovirus is verified for structural integrity of the transgene cassette consisting of the hTERT promoter linked to the candidate E1a replacement gene. They are then passaged to recover infectious adenovirus on producer cells. The producer cells are 293 cells, which are permissive for replication-defective (E1 negative) adenovirus; and A549 cells, which are non-permissive for E1 negative adenovirus. Adenoviral clones that are able to replicate in both A549 cells and 293 cells are selected for confirmation of the appropriate transgene insert. Clones that grow only on 293 cells but not A549 cells are excluded from the analysis except as negative controls. This assay is then used as a general screen for genes that complement for the E1a gene in allowing adenovirus replication.

The specificity of each new virus is tested by evaluating its ability to infect a panel of 5-10 tumor cell lines and normal cells, as in Example 2. Viruses that infect and kill telomerase-positive cells but not non-malignant cells are then tested in local and metastatic animal models of human tumors.

### Example 4

Transfection of Cells with YB-1

An adenovirus vector was constructed containing the human YB-1 encoding region under control of the CMV enhancer in place of the E1 region, with the objective that YB-1 would obviate or replace the requirement for E1 function and support replication of the virus in its absence. The virus was constructed using the AdMax™ recombinase-mediated adenovirus shuttle vector assembly system available from AdVec, Toronto Canada.

A549 cells were seeded approximately 12 h before infection in parallel with 293 cells as a replication control for the E1 deleted virus. A549 is a cell line derived from a human lung carcinoma (DSMZ ACC 107) which has high endogenous expression of the human YB-1 gene that should supplement YB-1 expression from the virus. On day 2, the cells were infected in duplicate at MOI 100, 1000, and 10,000. Two other adenovirus E1-deleted constructs (AdCAG-GFP and AdCAG-hTERT) served as negative controls.

At 24 hours after infection, the YB-1 construct caused complete lysis of A549 cells at MOI 1000 and 10,000. A549 cells infected at MOI 100 showed some toxicity (75-80% viable cells). In the MOI 1000 sample tested by RT-PCR for YB-1 mRNA expression, there was approximately 5.4 fold increase over endogenous YB-1 levels after 24 h. Taqman™ quantitative PCR of the YB-1 encoding DNA (a test for viral replication) 24 h after transduction at MOI 10,000 provided no direct evidence of DNA amplification above background, but the background was high due to the large number of viral particles used for transduction. At MOI 100, in the cells surviving for 6 days there was a ˜36-fold increase in DNA. AdCAG-GFP and AdCAG-hTERT (negative controls) showed negligible toxicity at MOI 10,000 at 24 h, and no observed toxicity at the lower MOI. Thus, killing of the A549 targets was only accomplished when YB-1 replaced E1a. All virus constructs did replicate and amplify in 293 cells, as expected, since these cells have endogenous E1a activity (positive control).

When MOI were titered between MOI 100 and MOI 1000 in subsequent experiments, toxicity at 24 h was ˜50% at MOI 1000, and negligible at MOI 100 in a dose-dependent fashion.

### Example 5

Reducing Tumor Growth in Vivo Using a TERT-driven Oncovirus

This experiment illustrates efficacy testing of a replication-conditional cytolytic virus in an animal model. The virus used was an oncolytic adenovirus in which the E1a gene is placed under control of the hTERT promoter (Example 2). The 143B cell line is a human osteosarcoma, and was obtained from the ATCC.

Six to eight week old female BALB/c nude (nu/nu) mice were injected subcutaneously in the flank with 2×10⁵ 143B cells with a Matrigel® support (Becton Dickinson). Tumors of ˜50 mm³ formed at the injection site by the 10^{th} day. The tumors were directly injected with the oncolytic virus in a volume of ˜50 μL daily from day 11 to day 15. Tumor size was monitored thereafter, and calculated assuming the shape of an ellipsoid body (L×W×H÷2).

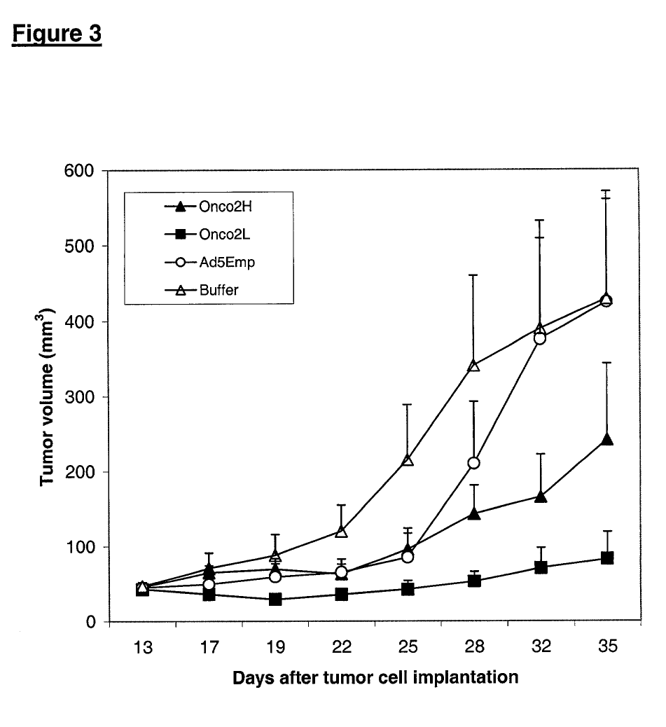

FIG. 3 shows growth of tumors in these animals as a function of days after engrafting the 143B cells. Onco2H=oncolytic virus at a dose of 2.5×10⁸ pfu per mouse. Onco2L=oncolytic virus at a dose of 1×10⁸ pfu per mouse. Ad5Emp=adenovirus vector lacking E1a and E3 genes. Buffer=buffer alone (negative control). n≈10 mice in each group.

The combined results of these experiments demonstrate that an adenovirus construct that is replication-conditional under control of the hTERT promoter specifically kills cancer cells and slows the rate of tumor growth by about 2-fold to over 5-fold, depending on dose.

It will be recognized that the compositions and procedures provided in the description can be effectively modified by those skilled in the art without departing from the spirit of the invention embodied in the claims that follow.

### SEQUENCE DATA

TABLE 2
Sequences listed in this Disclosure

| SEQ. ID NO: | Designation | Reference |
|---|---|---|
| 1 | Lambda clone designated λGφ5 | GenBank Accession AF121948 |
| | (ATCC Accession No. 98505) | International Patent Publication |
| | | WO 00/46355. |
| | Contains human Telomerase Reverse | |
| | Transcriptase (hTERT) genomic insert | |
| | (residues 44-15375), including the hTERT | |
| | promoter and upstream sequence. The | |
| | ATG translation initiation site begins at | |
| | residue 13545. | |
| 2 | Human YB-1 Y Box Binding Protein | GenBank Accession J03827.1 GI:340418 |
| | encoding DNA sequence | |
| | | Didier et al., Proc. Natl. Acad. Sci. USA |
| | | 85:7322, 1988. |
| | | See also GenBank P16991 GI 6136595 and |
| | | Singal et al., Gene 154:299, 1995. |
| 3 | Human YB-1 Y Box Binding Protein | Supra-CDS = 127-1080 |
| | amino acid sequence | |
| 4 | Human cytomegalovirus major immediate- | GenBank Accession M21295.1 GI:330620 |
| | early protein gene | |
| | | Akrigg et al., Virus Res. 2, 107, 1985 |
| | | See also Stenberg et al., J. Virol. 56, 665, |
| | | 1985 |

What is claimed as the invention is:

1. A replication-conditional virus with a genome comprising adenovirus replication genes and at least one heterologous gene that replaces a function of the adenovirus E1a gene.

2. The virus of claim 1, which is a cytolytic virus.

3. The virus of claim 1, wherein the heterologous gene is selected from Y-box transactivators, the immediate early genes of cytomegalovirus (CMV), and the oncogenes of human papillomavirus (HPV).

4. The virus of claim 3, wherein the heterologous gene is YB-1.

5. The virus of claim 3, wherein the heterologous gene is CMV IE1 or CMV IE2.

6. The virus of claim 3, wherein the heterologous gene is HPV E6, or HPV E7.

7. The virus of claim 1, wherein the heterologous gene (or another gene required for replication or assembly of the virus) is under control of a tissue or tumor specific transcriptional control element.

8. The virus of claim 7, wherein the transcriptional control element is a tissue specific promoter, which is a promoter for albumin, α-fetoprotein, prostate-specific antigen (PSA), mitochondrial creatine kinase (MCK), myelin basic protein (MB), glial fibrillary acidic protein (GFAP), or neuron-specific enolase (NSE).

9. The virus of claim 7, wherein the transcriptional control element is a tumor specific promoter, which is a promoter for telomerase reverse transcriptase (TERT), carcinoembryonic antigen (CEA), hypoxia-responsive element (HRE), Grp78, L-plastin, or hexokinase II.

10. The virus of claim 9, wherein the promoter comprises at least 25 consecutive nucleotides in SEQ. ID NO:1.

11. A host cell containing the virus of claim 1.

12. A method for selecting a virus according to claim 1, comprising transducing a host cell with a virus lacking an adenovirus gene required for replication or assembly, but comprising a heterologous gene; and determining whether replicated virus is produced by the cell

13. A method for killing a cancer cell, comprising contacting the cell with the virus of claim 7.

14. A method for killing a cell expressing telomerase reverse transcriptase (TERT), comprising contacting the cell with the virus of claim 10.

15. The method of claim 13, wherein the cancer is lung cancer, pancreatic cancer, medulloblastoma, cervical carcinoma, fibrosarcoma, or osteosarcoma.
